(19) United States
(12) Patent Application Publication
(10) Pub. No.: US 2014/0134645 A1
(43) Pub. Date: May 15, 2014

(54) METHOD OF ISOLATING OR COUNTING TARGET CELLS BY USING PHOTOCLEAVABLE LINKER COUPLED WITH FLUORESCENT DYE

(71) Applicant: Samsung Electronics Co., Ltd., Suwon-si (KR)

(72) Inventors: Hun-joo LEE, Hwaseong-si (KR); Jin-ho OH, Seoul (KR); Seung-hyun LEE, Suwon-si (KR); Jong-myeon PARK, Incheon (KR)

(21) Appl. No.: 13/916,017

(22) Filed: Jun. 12, 2013

(30) Foreign Application Priority Data
Nov. 14, 2012 (KR) 10-2012-0129099

(51) Int. Cl. G01N 33/58

(52) U.S. Cl. CPC G01N 33/582
USPC 435/7.23; 435/7.1; 435/7.4

(57) ABSTRACT

A method of isolating or counting target cells using photocleavable linkers coupled with fluorescent dyes, for qualitative or quantitative analysis of protein, gene analysis, and/or morphological analysis after removing the dye.

### CROSS-REFERENCE TO RELATED APPLICATIONS

This application claims the benefit of Korean Patent Application No. 10-2012-0129099, filed on Nov. 14, 2012, in the Korean Intellectual Property Office, the disclosure of which is incorporated herein in its entirety by reference.

### BACKGROUND

1. Field

The present disclosure relates to a method of isolating or counting target cells by using photocleavable linker coupled with fluorescent dye.

2. Description of the Related Art

Cells are the basic units of the human body, and have different shapes in different organs. Generally, diseases are diagnosed by conducting a tissue biopsy test, but as the accuracy of cell tests has improved recently, simple and accurate diagnosis of diseases has been made possible. Cells in solid tissues may be isolated via microscopic observation at their locations, while cells in blood may not be easily selectively isolated because blood is a complex of a variety of cells. Isolating only a target cell from a sample, like blood, containing a variety of cells with different characteristics, or removing undesirable cells from the sample, is essential for a cell count, understanding of the shapes and characteristics of cells, identifying surface or intracellular proteins of cells via an immunoassay, single cell analysis, and/or gene analysis.

Circulating tumor cells (CTCs) are a type of tumor cells present in a very small amount in the blood of a metastatic cancer patient. CTCs may be identified in a patient before any tumor is initially detected from the patient. In some cases, CTCs may be also found even after surgery is performed to remove cancer cells. Thus, detection or isolation of CTCs, or analysis of isolated CTCs, may have a crucial role in early cancer diagnosis, early cancer metastasis diagnosis, and prediction of the chances of recurrence.

Generally, cell isolation is performed by using various expressed proteins included in target cells. However, since certain proteins also exist in other cells as well as in the target cells, isolation processes are required to be repeated several times. In this regard, a later isolation process may be affected by dye used in previous processes, and thus precise isolation may not be possible in the repeated isolation processes. Therefore, a method of effectively isolating target cells included in a biological sample is needed.

### SUMMARY

Provided is a method of isolating target cells comprising contacting a dye complex with a sample including target cells to form dye complex-bound target cells, wherein the dye complex includes (a) a material binding to a target material, (b) a cleavable linker that is linked to the material binding to the target material, and (c) a fluorescent dye that is coupled with the cleavable linker; and isolating the resulting dye complex-bound target cells.

Provided is a method of counting target cells comprising contacting a dye complex with a sample including target cells to form dye complex-bound target cells, wherein the dye complex includes (a) a material binding to a target material, (b) a cleavable linker that is linked to the material binding to the target material, and (c) a fluorescent dye that is coupled with the cleavable linker; and measuring a signal from the dye complex-bound target cells.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings.

The patent or application file contains at least one drawing executed in color. Copies of this patent or patent application publication with color drawing(s) will be provided by the Office upon request and payment of the necessary fee.

FIG. 1 is a schematic view illustrating a process of continuous isolation of cells by using an antibody-PC linker-fluorescent dye complex, according to an embodiment of the present invention.

FIG. 2A shows a merged image of FIG. 2C-E. FIG. 2B shows an image of SK-BR3 before staining. FIG. 2C shows a DAPI fluorescent image of SK-BR3 that is not bound with the complex of FIG. 1. FIG. 2D and 2E show a fluorescent image of SK-BR3 that is bound with an anti-cytokeratin 7, 8, 18 antibody-PC linker-FITC complex, and a fluorescent image of SK-BR3 that is bound with an anti-EpCAM antibody-PC linker-Rhodamine complex, respectively.

FIG. 3 shows a fluorescent signal change of FITC and Rhodamine dye bound to breast cancer cells BT474 according to light exposure.

FIG. 4 shows the result of quantitatively comparing an staining efficiency of cells after primary staining, light exposure, and secondary staining; and an staining efficiency of cells after primary staining as a control. Fluorescence intensity is indicated on the y-axis.

FIGS. 5A-C illustrate a cell mixture isolated by using fluorescence-activated cell sorting (FACS) after reacting the cell mixture with an anti-CD45 antibody-FITC complex and an anti-EGFR antibody-PC linker-Rhodamine complex. FIG. 5A shows cells before the isolation, FIG. 5B shows cells bound with the anti-EGFR antibody-PC linker-Rhodamine complex after the isolation, and FIG. 5C shows the cells bound to the anti-CD45 antibody-FITC complex after the isolation.

FIGS. 6A-C illustrate a cell mixture secondarily isolated after reacting the mixture with an anti-CD45 antibody-FITC complex and an anti-EGFR antibody-PC linker-Rhodamine complex, primarily isolating the cells by using FACS, exposing them to light, and reacting them with an anti-HER2 antibody-PC linker-Rhodamine complex. FIG. 6A is a fluorescent microscopic image of white blood cells (WBCs) bound with the anti-CD45 antibody-FITC complex. FIG. 6B is a fluorescent microscopic image of a cell mixture of MDA-MB-231 and BT474 bound with the anti-EGFR antibody-PC linker-Rhodamine complex. FIG. 6C is a fluorescent microscopic image of BT474 cells bound to the anti-HER2 antibody-PC linker-Rhodamine complex.

FIG. 7 illustrates a process to prepare an antibody-PC linker-FITC complex. First, 2-methylcyclopent-4-ene-1,3-dione group of heterobifunctional photocleavable linker is conjugated to fluorescein PEG Thiol. Then, pyrrolidine-2,5-dione group of PC linker-FITC is conjugated to amino group of the antibody.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

According to an embodiment of the present invention, a method of isolating target cells comprises, contacting a dye complex with a sample including target cells to form dye complex-bound target cells; and then isolating the dye complex-bound target cells. The dye complex comprises, consists essentially of, or consists of (a) a material binding to the target material, (b) a cleavable linker that is linked to (e.g., bound to or coupled with) the material binding to the target material, and (c) a fluorescent dye that is linked to (e.g., bound to or coupled with) the cleavable linker.

The contacting of the dye complex with the sample comprising target cells may be performed under conditions that induce binding between the material binding to the target material of the dye complex and the target material present on or in the cell included in the sample. For example, the contacting may be performed under conditions that induce specific binding between an antibody and an antigen. Exemplary temperatures for the contacting step include 15° C. to 30° C., 18° C. to 27° C., or 20° C. to 25° C. (e.g., 15° C., 16° C., 17° C., 18° C., 19° C., 20° C., 21° C., 22° C., 23° C., 24° C., 25° C., 26° C., 27° C., 28° C., 29° C., or 30° C.). Exemplary pH values for the contacting step include 6 to 8, 6.2 to 7.8, or 6.5 to 7.5.

The dye complex may be a plurality of dye complexes including multiple materials (e.g., antibodies) that bind to the same or different target material and multiple fluorescent dyes. Each fluorescent dye of the plurality of dye complexes may be selected in such a manner that an overlap among emission spectrum of each fluorescent dye is minimized. The number of different dye complexes may be 2 to 20 (i.e., 2, 3, 4, 5, 6, 7, 8, 9, 10, 11, 12, 13, 14, 15, 16, 17, 18, 19, or 20) and preferably 2, 3, or 4.

In each of the dye complexes, the material binding to a target material may be, for example, selected from the group consisting of an antibody, an antigen, an aptamer, a receptor, a ligand, an enzyme substrate, an enzyme inhibitor, an enzyme cofactor, and an enzyme. The target material may be, for example, a material that enables the discrimination of cancer cells from other types of cells in a sample. For example, the target material may be selected from the group consisting of a protein, sugar, lipid, nucleic acid, and any combination thereof. The target material may be present on a surface of a cell or in a cell. For example, the target material may be cytokeratin, which is a protein existing in a cell, or EpCAM, IGFR, EGFR, HER2, which are proteins existing on a surface of a cell.

The cleavable linker may be, for example, a photocleavable linker. The photocleavable linker may be cleaved when irradiated with a UV ray or an X-ray. For example, the photocleavable linker may be a compound including a 2-nitrobenzyl group and a (coumarin-4-yl)methyl group.

The fluorescent dye may be, for example, selected from the group consisting of FITC, Alexa Fluor 488, GFP, CFSE, CFDA-SE, DyLight 488, PE, PI, PerCP, PerCP-Cy5.5, PE-Alexa Fluor 700, PE-Cy5 (TRI-COLOR), PE-Cy5.5, PE-Alexa Fluor 750, PE-Cy7, APC, APC-Cy7, APC-eFluor 780, Alexa Fluor 700, Cy5, Draq-5, Pacific Orange, Amine Aqua, Pacific Blue, DAPI, Alexa Fluor 405, eFluor 450, eFluor 605 Nanocrystals, eFluor 625 Nanocrystals, and eFluor 650 Nanocrystals. For example, the fluorescent dye may be selected from the group consisting of FITC, DAPI, Cy5, Cy3, Texas Red, and Rhodamine.

The sample may be any biological sample in which cells may exist and may be, for example, selected from the group consisting of a biopsy sample, a tissue sample, a cell suspension including separated cells suspended in a liquid medium, a cell culture, and any combination thereof. In one embodiment, the biological sample is isolated from an animal, such as a primate (e.g., human), mouse, rat, guinea pig, hamster, rabbit, cat, dog, pig, cow, or horse.

The sample may be an animal body fluid and may be, for example, selected from the group consisting of blood, marrow fluid, lymphatic fluid, saliva, lachrymal fluid, urine, mucous fluid, amniotic fluid, and any combination thereof. For example, in order to separate circulating tumor cells (CTCs), blood may be used as the biological sample. The sample may be a cell mixture including different types of cells mixed therein. The mixture may include cells having the target material and other cells that may exist in the biological sample. The target cells included in the sample may be, for example, selected from the group consisting of a CTCs, cancer stem cells, immunocytes, fetal stem cells, fetal cells, cancer cells, and tumor cells.

The contacting of the sample comprising target cells with the dye complex may be performed in a solution including the sample. The solution provides an environment where the sample and the complex may react stably, and the solution may be a buffer solution known in the art. The solution may be, for example, phosphate buffered saline (PBS) or phosphate buffered saline with Tween 20 (PBST).

Before the contacting of the sample with the dye complex, the method of isolating target cells according to an embodiment of the present invention may include, for example, fixing the target cells (e.g., by adding chemical fixatives such as aldehydes for crosslinking including paraformaldehyde as described in Example 2, alcohols as precipitating fixatives, oxidizing agents, mercurials, and picrates), increasing cell permeability (e.g., by adding organic solvents, such as methanol and acetone, or detergents such as Triton-X 100 as described in Example 2, saponin, and Tween-20), and/or blocking to reduce non-specific reactions (e.g., by adding bovine serum albumin as described in Example 2, goat serum, fish skin gelatin, horse serum, swine serum, donkey serum, or rabbit serum).

The isolating of the dye complex-bound target cells may include, for example, isolating the dye complex-bound target cells by flow cytometry. The flow cytometry method may be, for example, fluorescence-activated cell sorting (FACS).

The method of isolating target cells according to the present embodiment may further comprise irradiating light on the isolated dye complex-bound target cells to cleave the cleavable linker. The wavelength of the irradiated light used in the method may vary depending on a type of the linker. For example, the wavelength may be from about 100 nm to about 600 nm (e.g., 150 nm, 200 nm, 250 nm, 300 nm, 350 nm, 400 nm, 450 nm, 500 nm, or 550 nm), or from about 340 nm to about 370 nm (e.g., 345 nm, 350 nm, 355 nm, 360 nm, or 365 nm), for example, about 365 nm. The irradiating of the light may result in, for example, the removal of a remaining part of the linker and the fluorescent dye other than the antibody that is specifically bound to the target material and a part of the linker in the dye complex that is bound to the target cells.

In some embodiments, after the irradiating of light is performed, the method of isolating target cells may further comprise contacting a dye complex with a sample including target cells to form dye-complex-bound target cells, and isolating the dye complex-bound target cells. In some embodiments, after the irradiating of light is performed, a cycle comprising contacting a dye complex with the sample including the target cells to form dye complex-bound target cells, isolating the dye complex-bound target cells from a resulting product, and irradiating light on the isolated dye complex-bound target cells to cleave the cleavable linker may be sequentially repeated at least once. In some embodiments, after the at least one sequentially repeated cycle, the method of isolating target cells may further comprise contacting a dye complex with a sample including target cells to form dye-complex-bound target cells, and isolating the dye complex-bound target cells. As described above, the dye complex comprises (a) a material binding to a target material, (b) a cleavable linker that is linked to the material binding to the target material, and (c) a fluorescent dye that is coupled with the cleavable linker.

In each cycle, the dye complex used may be the same as or different than the dye complex that was used in a previous cycle. Preferably, the dye complex has a material binding to a target material which is different from that in the dye complex used in the previous cycle. For example, the dye complex can include a second antibody having antigen specificity different from a first antibody used in a previous cycle. The fluorescent dye of the dye complex of each cycle may be the same as or different than the dye included in the dye complex of the previous cycle. Also, the dye complex of each cycle may be a plurality of dye complexes including a different material that binds to a different target material and a different fluorescent dye. Each fluorescent dye of the plurality of dye complexes may be selected in such a manner that an overlap among emission spectrum of each fluorescent dye is minimized. The number of different dye complexes may be 2 to 20, for example, 2, 3, or 4.

According to another embodiment of the present invention, a method of counting target cells comprises, contacting a dye complex with a sample including target cells to form dye complex-bound target cells, and measuring a signal from the dye complex-bound target cells. The dye complex comprises (a) a material binding to a target material, (b) a cleavable linker that is linked to the material binding to a target material, and (c) a fluorescent dye that is coupled with the cleavable linker.

The contacting the dye complex with the sample may be performed in the same manner as described above. The material binding to the target material, the cleavable linker, and the fluorescent dye are as described above. The sample is as described above.

The measuring of the signal may include, for example, counting cells by measuring a signal generated from the fluorescent dye of the dye complex-bound target cells. The measuring may be performed, for example, by flow cytometry. The flow cytometry method may be, for example, FACS.

The present invention will be described in further detail with reference to the following examples. These examples are for illustrative purposes only and are not intended to limit the scope of the present invention.

### EXAMPLE 1

### Cancer Cell Labeling by using Antibody-Photocleavable (PC) Linker-Fluorescent Dye Complex

10 μL of 50 nmol heterobifunctional photocleavable linker (self-manufactured, see formula below) in DMF and 15 μL of 50 nmol dye solution (Fluorescein PEG Thiol (MW 5000), Rhodamine PEG Thiol (MW 5000), Nanocs Inc.) in 50 mM phosphate buffer (pH 5) were reacted for 30 minutes at room temperature.

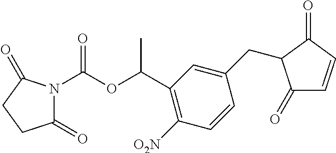 [SMILES: CC(OC(=O)N1C(=O)CCC1=O)c1cc(CC2C(=O)C=CC2=O)ccc1[N+](=O)[O-]]

100 μL of 10 nmol anti-cytokeratin 7, 8, 18 antibody or anti-EpCAM antibody in 1×PBS was added, and the mixture was reacted overnight at 4° C. Then, unreacted dye was removed by using an Amicon® Ultra centrifugal filter (Millipore, MW 30,000), and the resulting product was concentrated five-fold. 5 μL of the obtained antibody-PC linker-dye complex was added to a PBS buffer solution with 1% BSA, and breast cancer cells SK-BR3 were added into the mixture and stirred at a rate of 15 rpm for 1 hour. Binding between SK-BR3 and the complex was confirmed by a fluorescent microscope (Olympus IX81).

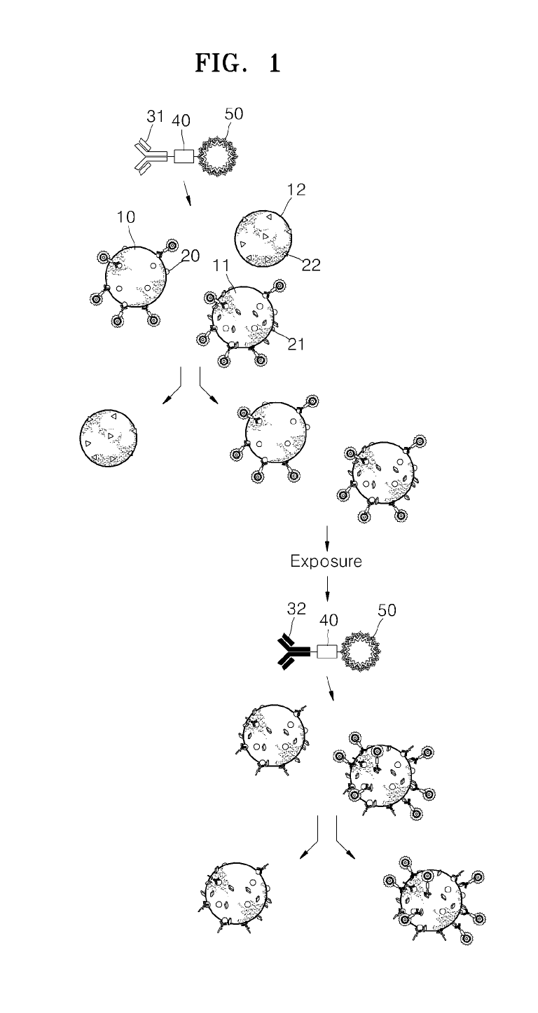

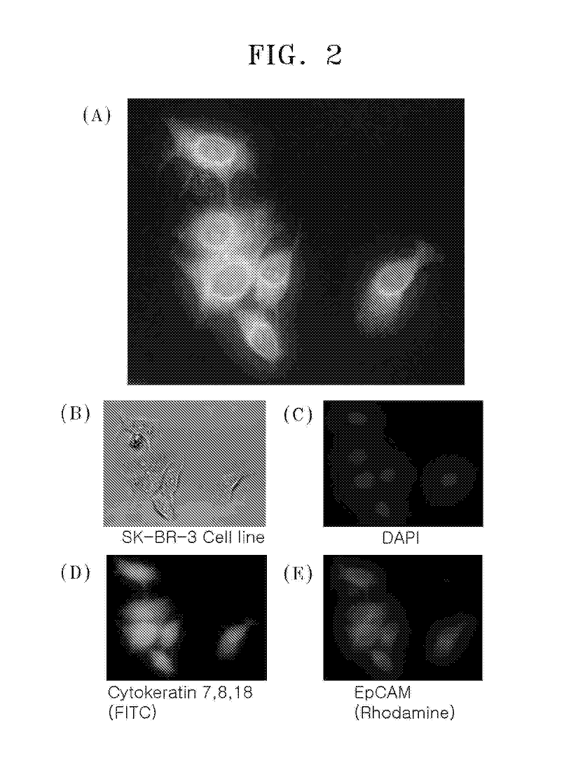

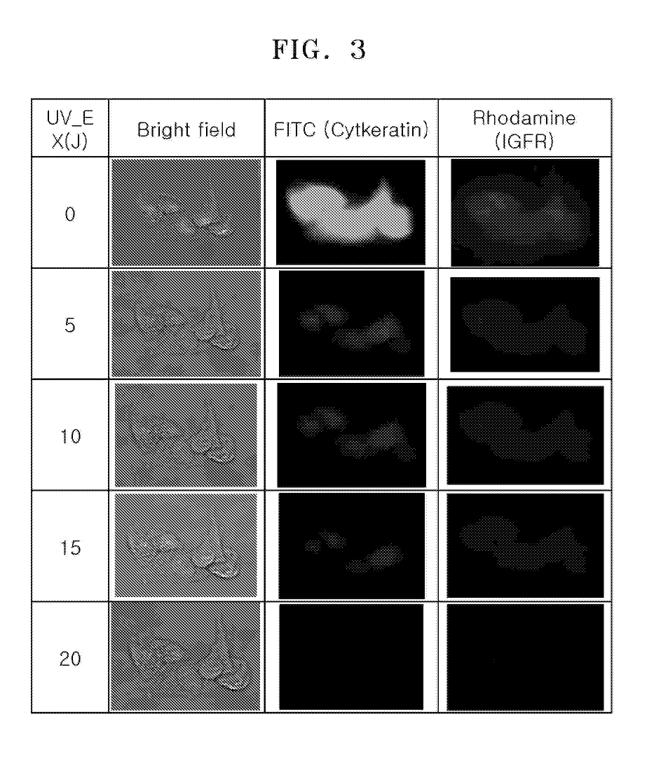

FIG. 2A shows a merged image of FIG. 2C-E. FIG. 2B shows an image of SK-BR3 before staining. FIG. 2C shows a DAPI fluorescent image of SK-BR3 that is not bound with the complex of FIG. 1. FIGS. 2D and 2E show a fluorescent image of SK-BR3 that is bound with the anti-cytokeratin 7, 8, 18 antibody-PC linker-FITC complex, and a fluorescent image of SK-BR3 that is bound with the anti-EpCAM antibody-PC linker-Rhodamine complex. It was confirmed that the cancer cells were labeled by the added complex, respectively.

### EXAMPLE 2

### Verification of Dye Dissection According to Light Exposure

Breast cancer cells BT474 were fixed with 4% paraformaldehyde for 10 minutes, and treated with 0.2% Trition-X 100 for 10 minutes to increase permeability of the cells. After adding 1% BSA solution, the cells were stained with an anti-cytokeratin antibody-PC linker-FITC complex or an anti-IGFR antibody-PC linker-Rhodamine complex for 60 minutes at room temperature. Then, the cells were washed with 1×PBS and observed under a fluorescent microscope while increasing an intensity of irradiation (J) of light at a wavelength of 365 nm.

FIG. 3 shows a fluorescent signal change of FITC and Rhodamine dye bound to the breast cancer cells BT474 according to light exposure. It was confirmed that as the intensity of irradiation increased, an intensity of the fluorescent signal decreased, and accordingly a ratio of dye being removed increased.

### EXAMPLE 3

### Staining Reset Cells

Breast cancer cells SK-BR3 were fixed with 4% paraformaldehyde for 10 minutes, and treated with 0.2% Trition-X 100 for 10 minutes to increase permeability of the cells. After adding 1% BSA solution, the cells were stained with an anti-cytokeratin antibody-PC linker-FITC complex or an anti-IGFR antibody-PC linker-Rhodamine complex, respectively, for 60 minutes at room temperature. Then, the cells were washed with 1×PBS, exposed to light (20 J) at 365 nm, followed by staining the cells with the anti-EpCAM antibody-PC linker-FITC complex or the anti-EGFR antibody-PC linker-Rhodamine complex, respectively, for 60 minutes at room temperature, and measuring fluorescence intensity of the cells according to each dye complex. Cells of a control group were treated in the same manner as described above in regard to fixing the cells, increasing permeability of the cells, and BSA blocking, followed by staining the cells with an anti-EpCAM antibody-PC linker-FITC complex or an anti-EGFR antibody-PC linker-Rhodamine complex, respectively, for 60 minutes at room temperature, and measuring fluorescence intensity of the cells according to each dye complex.

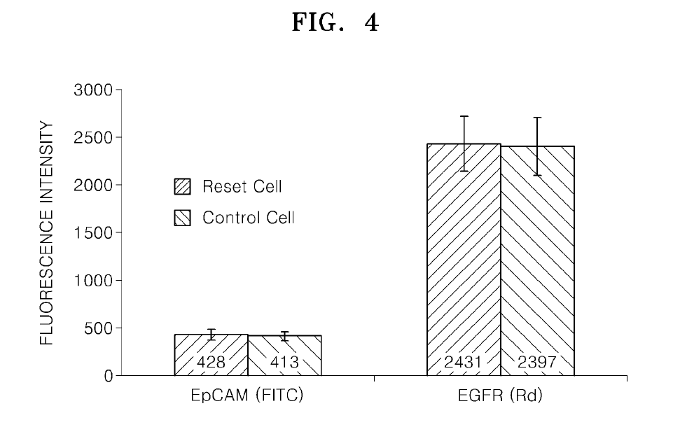

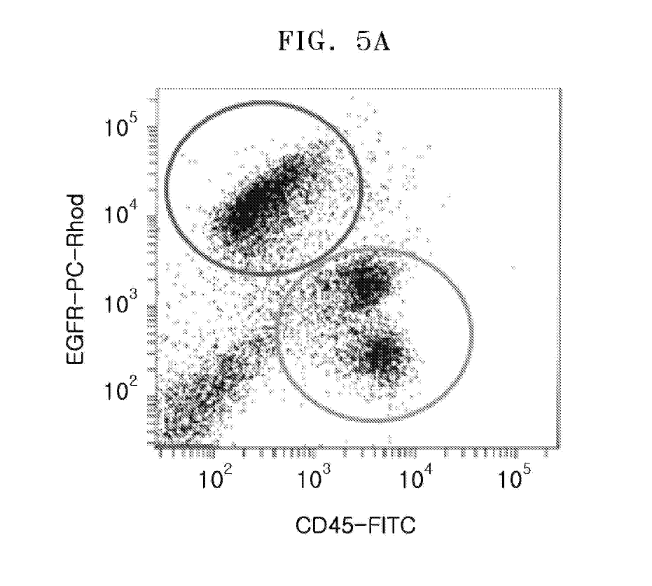

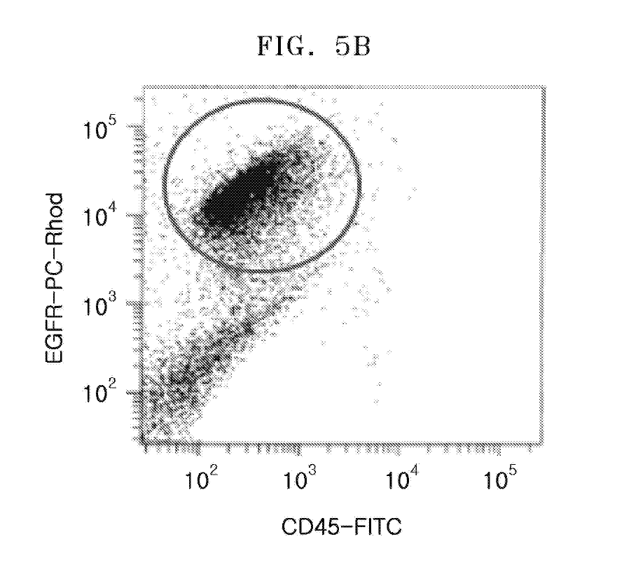

FIG. 4 shows the result of quantitatively comparing staining efficiency between the cells after primary staining, light exposure, and secondary staining; and the cells after primary staining as the control group. In both cases of staining with either anti-EpCAM antibody-PC linker-FITC complex or the anti-EGFR antibody-PC linker-Rhodamine complex, fluorescence intensity of the secondary staining after the light exposure showed an equal level with fluorescence intensity of the primary staining, within an error range. Therefore, it was confirmed that light exposure does not influence the cell staining.

### EXAMPLE 4

### Flow Cytometric Analysis

An anti-CD45 antibody-FITC complex and the anti-EGFR antibody-PC linker-Rhodamine complex obtained in Example 1 were added to a cell solution including white blood cells (WBCs), MDA-MB-231 cells, and BT474 cells in 1×PBS, and reacted for 60 minutes at room temperature to stain the cells. Then, the cells were washed with 1×PBS, and flow cytometric analysis was performed by using a FACS apparatus (BD FACSAria III™ Cell Sorter). A laser of 488 nm, a 585/42 filter, and a 556 longpass (LP) mirror were used. MDA-MB-231 cells and BT474 cells bound with the anti-EGFR antibody-PC linker-Rhodamine complex were isolated from the WBCs bound with the anti-CD45 antibody-FITC. Next, the isolated MDA-MD-231 cells and BT474 cells were exposed to light at a wavelength of 365 nm (20 J) to remove the bound Rhodamine dye. A mixture of the two isolated cells was washed with 1X PBS, and an anti-HER2 antibody-PC linker-Rhodamine complex was added, and then reacted for 60 minutes at room temperature to stain the cells. Then, BT474 cells were isolated by using FACS.

FIG. 1 is a schematic view illustrating a process of continuous isolation of cells using an antibody-PC linker-fluorescent dye complex according to an embodiment of the present invention. A fluorescent dye 50 may be coupled with a cleavable linker 40. A terminus of the cleavable linker 40 may be coupled with a material binding to a target material, such as an antibody 31, and able to specifically bind to target materials 20 of target cells 10 and 11. After a first isolation, the isolated cells are exposed to light, then another dye complex comprising another material binding to another target material, such as another antibody 32, may be able to specifically bind to other target materials 21 of the target cell 11, and thus a second isolation may be performed.

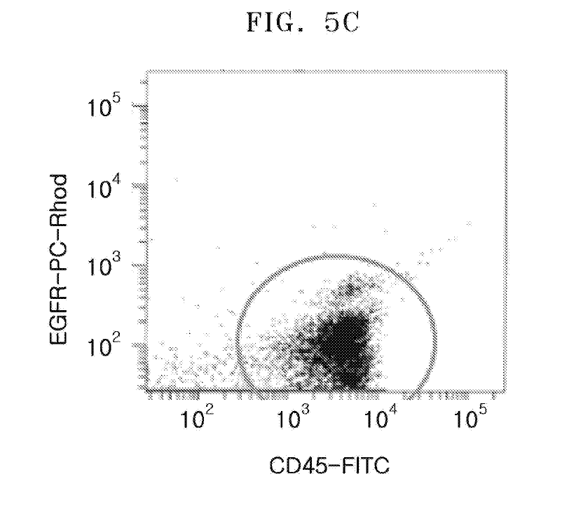

FIGS. 5A-C illustrate the result of isolating the cells by using FACS after reacting the cell mixture with the anti-CD45 antibody-FITC and anti-EGFR antibody-PC linker-Rhodamine complex. FIG. 5A shows the cells before the isolation, FIG. 5B shows the cells bound with the anti-EGFR antibody-PC linker-Rhodamine complex after the isolation, and FIG. 5C shows the cells bound to the anti-CD45 antibody-FITC complex after the isolation. It was confirmed that the cell isolation was sufficiently performed with the anti-PC linker-fluorescent dye complex.

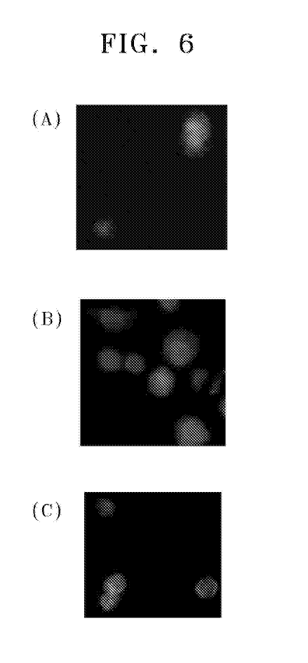

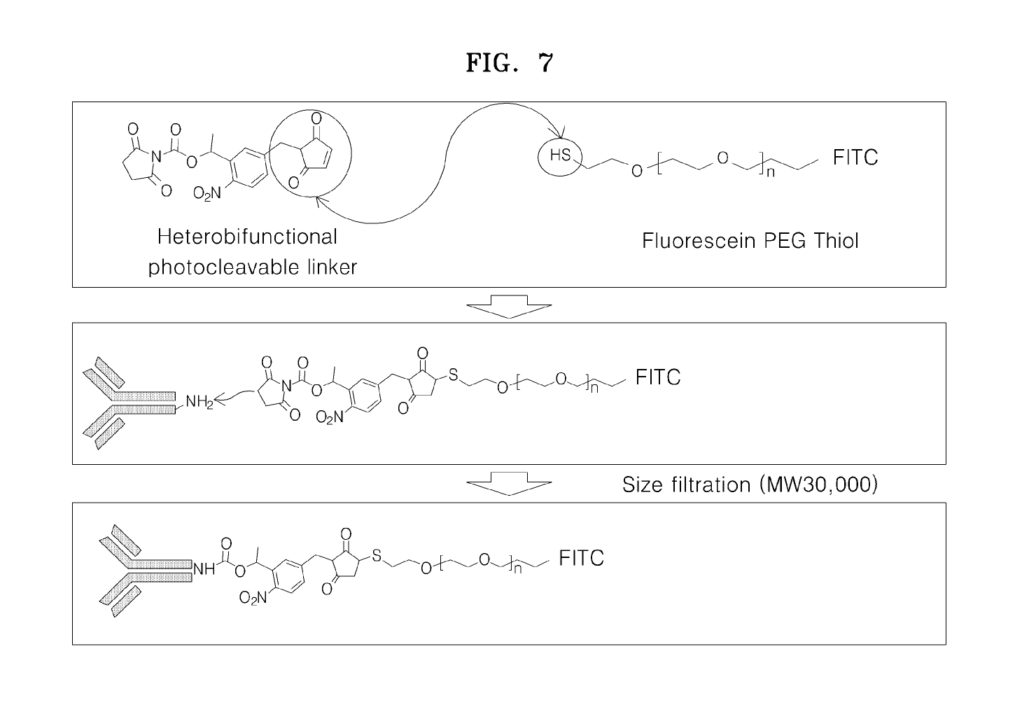

FIGS. 6A-C illustrate a cell mixture secondarily isolated after reacting the mixture with an anti-CD45 antibody-FITC complex and an anti-EGFR antibody-PC linker-Rhodamine complex, primarily isolating the cells by using FACS, exposing them to light, and reacting them with an anti-HER2 antibody-PC linker-Rhodamine complex. FIG. 6A is a fluorescent microscopic image of white blood cells (WBCs) bound with the anti-CD45 antibody-FITC complex. FIG. 6B is a fluorescent microscopic image of a cell mixture of MDA-MB-231 and BT474 bound with the anti-EGFR antibody-PC linker-Rhodamine complex. FIG. 6C is a fluorescent microscopic image of BT474 cells bound to the anti-HER2 antibody-PC linker-Rhodamine complex.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of isolating target cells, the method comprising:
contacting a dye complex with a sample including target cells to form dye-complex-bound target cells, wherein the dye complex comprises (a) a material binding to a target material, (b) a cleavable linker that is linked to the material binding to the target material, and (c) a fluorescent dye that is coupled with the cleavable linker; and
isolating the dye complex-bound target cells.

2. The method of claim 1, wherein the material binding to a target material is an antibody, an antigen, an aptamer, a receptor, a ligand, an enzyme substrate, an enzyme inhibitor, an enzyme cofactor, or an enzyme.

3. The method of claim 1, wherein the cleavable linker is a photocleavable linker.

4. The method of claim 1, wherein the fluorescent dye is FITC, DAPI, Cy5, Cy3, Texas Red, or Rhodamine.

5. The method of claim 1, wherein the target material is a protein, sugar, lipid, nucleic acid, or any combination thereof.

6. The method of claim 1, wherein the target cells are circulating tumor cells (CTCs), cancer stem cells, immunocytes, fetal stem cells, fetal cells, cancer cells, or tumor cells.

7. The method of claim 1, wherein isolation of the target cells is performed by flow cytometry.

8. The method of claim 3, further comprising irradiating light on the isolated dye complex-bound target cells to cleave the cleavable linker.

9. The method of claim 8, further comprising contacting a dye complex with a sample including target cells to form dye-complex-bound target cells, and isolating the dye complex-bound target cells.

10. The method of claim 8, wherein the method further comprises at least one sequentially repeated cycle after the irradiation of the isolated dye complex-bound target cells to cleave the cleavable linker,
wherein the at least one sequentially repeated cycle comprises contacting a dye complex with a sample including target cells to form dye-complex-bound target cells, isolating the dye complex-bound target cells, and irradiating light on the isolated dye complex-bound target cells to cleave the cleavable linker.

11. The method of claim 10, further comprising contacting a dye complex with a sample including target cells to form dye-complex-bound target cells, and isolating the dye complex-bound target cells.

12. The method of claim 10, wherein each cycle comprises the use of a dye complex that is different from the dye complex used in a previous cycle, with cells that were isolated in the previous cycle.

13. The method of claim 12, wherein the dye complex has a material binding to a target material which is different from that in the dye complex used in the previous cycle.

14. A method of counting target cells, the method comprising:
contacting a dye complex with a sample including target cells to form dye complex-bound target cells, wherein the dye complex comprises (a) a material binding to a target material, (b) a cleavable linker that is linked to the material binding to the target material, and (c) a fluorescent dye that is coupled with the cleavable linker; and
measuring a signal from the dye complex-bound target cells to count the target cells.

15. The method of claim 14, wherein the measuring of the signal is performed by flow cytometry.

16. The method of claim 14, wherein the material binding to a target material is an antibody, an antigen, an aptamer, a receptor, a ligand, an enzyme substrate, an enzyme inhibitor, an enzyme cofactor, or an enzyme.

17. The method of claim 14, wherein the cleavable linker is a photocleavable linker.

18. The method of claim 14, wherein the fluorescent dye is FITC, DAPI, Cy5, Cy3, Texas Red, or Rhodamine.

19. The method of claim 14, wherein the target material is a protein, sugar, lipid, nucleic acid, or any combination thereof.

20. The method of claim 14, wherein the target cells are circulating tumor cells (CTCs), cancer stem cells, immunocytes, fetal stem cells, fetal cells, cancer cells, or tumor cells.
